(19) United States
(12) Patent Application Publication
(10) Pub. No.: US 2020/0190464 A1
(43) Pub. Date: Jun. 18, 2020

(54) NOVEL LACTOBACILLUS HAVING ANTIVIRAL EFFECT AND COMPOSITION THEREOF

(71) Applicant: COTDE, INC., Chungcheongnam-do (KR)

(72) Inventors: DONG IL JANG, Chungcheongnamdo (KR); DAE HYUN KIM, Jeju-do (KR)

(21) Appl. No.: 16/632,508

(22) PCT Filed: Jun. 12, 2018
(86) PCT No.: PCT/KR2018/006618

§ 371 Date: Jan. 20, 2020

(30) Foreign Application Priority Data
Jul. 25, 2017 (KR) 10-2017-0093953

(51) Int. Cl. C12N 1/20; A23K 10/16; A23K 20/195; A23L 33/135; A61K 35/747

(52) U.S. Cl. CPC C12N 1/20; A23K 10/16; C12R 1/25; A23L 33/135; A61K 35/747; A23K 20/195

(57) ABSTRACT

A Lactobacillus paraplantarum CK401 having an antiviral effect, and more specifically to novel kimchi-derived microorganism Lactobacillus paraplantarum CK401 having inhibitory activity against influenza viruses, particularly avian influenza virus, and the use thereof. According to the present invention, any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular weight cutoff of 10 kD, have excellent antiviral activity, and thus may be usefully used for drugs, foods, fermented products, food additives, health supplements, feeds, feed additives, etc.

### BACKGROUND

The present invention relates to novel Lactobacillus paraplantarum CK401 having an antiviral effect, and more specifically to novel kimchi-derived microorganism Lactobacillus paraplantarum CK401 having inhibitory activity against influenza viruses, particularly avian influenza virus, and the use thereof.

Feed additive antimicrobial agents have been used at low levels for the treatment and prevention of diseases in food animals over the past 50 years, and have actually greatly contributed to improvement in the production of various kinds of livestock (weight gain, increased egg production rate, improved feed efficiency, increased survival rate, improved meat quality, improved fertility rate, etc.).

However, the abuse of antibiotics has led to increased levels of residual antibiotics in livestock products, which have seriously threatened consumer health. Accordingly, consumers have demanded livestock products that are safer and harmless to the human body. In addition, due to the emergence of super bacteria (vancomycin-resistant Staphylococcus aureus, VRSA) resistant to vancomycin which is the most potent among antibiotics developed to date, consumers' anxiety about antibiotic resistance has increased, and thus the pressure to refrain from using antimicrobial substances in the livestock industry has increased. Accordingly, as a preventive aspect of human medicine, countries such as Brazil and Thailand, which export livestock products, including chicken, to EU countries, are expected to reduce the use of various antimicrobial agents, which have been used as growth promoters, to the same level as in EU countries. Thus, authorities in many countries interested in it have reviewed the licensing, regulatory methods and certification processes for food additive antimicrobial agents to be used in livestock production, and have tended to tighten regulations, and also have actively conducted a review of risk assessments. This inevitably means that there is a market environment in which a market for antibiotic substitutes or feed additives that can prevent or treat livestock diseases may grow rapidly.

However, until now, feed additives capable of effectively treating or preventing antibiotic and antiviral diseases in livestock have not been developed, and some lactic acid bacteria have been used for the above purpose, but do not exhibit a significant effect.

In addition, in Korea since 2004, high pathogenic avian influenza occurred again in the winter of 2006 and early 2007, which caused great difficulties. Looking at the annual number of people infected with avian influenza and the annual number of deaths caused by avian influenza, it can be seen that avian influenza is not an annual disturbance, but is the threat of catastrophe. However, vaccines and treatments that can be effectively used for viral diseases at home and abroadhave been rarely developed. Although Tamiflu was developed and has been sold by Roche, a multinational pharmaceutical company, Tamiflu has problems in that it is very expensive and cannot be used for animals due to its limited production.

### SUMMARY OF THE INVENTION

The present invention has been conceived to overcome the above-described problems, and an object of the present invention is to provide a Tamiflu-replaceable natural antiviral agent capable of effectively treating or preventing viral diseases, particularly avian influenza viral disease, and a composition containing the same.

In order to accomplish the above object, the present invention provides novel microorganism Lactobacillus paraplantarum CK401 KCTC 13287BP having antiviral activity.

The present invention also provides an antiviral composition containing any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD.

The antiviral composition according to the present invention as described above may be any one or more compositions selected from the group consisting of a pharmaceutical composition, a health supplement composition, a food composition, a food additive composition, a feed composition, and a feed additive composition.

The present invention also provides a method for preventing and treating viral infection in animals other than humans, the method including administering, to animals other than humans, any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD.

The present invention as described above provides a composition and method showing antiviral activity against a wide range of viruses, and particularly provides a composition and method showing excellent activity against influenza viruses, particularly avian influenza viruses.

According to the present invention, any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD, have excellent antiviral activity, and thus may be usefully used for drugs, foods, fermented products, food additives, health supplements, feeds, feed additives, etc. In addition, they show excellent antiviral activity even when mass-produced, and thus are industrially very useful. In particular, they show strong activity against highly contagious influenza viruses, particularly avian influenza viruses.

### BRIEF DESCRIPTION OF DRAWINGS

FIG. 1 shows the titer of AI virusas a function of the dilution rate of CK401;

FIG. 2 shows the effective titer of AI virus as a function of the culture time (days) of CK401;

FIG. 3 shows the change in daily egg production rate of a PC farm after administering a test sample;

FIG. 4 shows the change in daily egg production rateof an SH farm after administering a test sample;

FIG. 4 shows the change in weekly egg production rate of a CW farm after administering a test sample; and

FIG. 6 shows the results of FT-IR analysis of a retentate on a membrane filter having a molecular cutoff of 10 kD.

### DETAILED DESCRIPTION OF THE INVENTION

The present invention is directed to Lactobacillus paraplantarum CK401 KCTC 13287BP.

The Lactobacillus paraplantarum CK401 KCTC 13287BP according to the present invention is a novel Lactobacillus strain isolated from kimchi. Molecular phylogenetic analysis was performed based on the 16S rRNA nucleotide sequence. As a result, the genome was at least 99% identical to that of the Lactobacillus paraplantarum DSM 10667 strain, and thus the Lactobacillus paraplantarum CK401 KCTC 13287BP was identified to be Lactobacillus paraplantarum. The Lactobacillus paraplantarum CK401 KCTC 13287BP was deposited with the Korean Collection for Type Cultures (KCTC),

Korea Research Institute of Bioscience and Biotechnology, on Jun. 13, 2017 under accession number KCTC13287BP.

Lactobacillus paraplantarum is a lactic acid bacterium belonging to the genus Lactobacillus strain, which appears during fermentation of kimchi, a product which is generally prepared by salting radish, cabbage, cucumber and the like or mixing the salted vegetable with seasonings such as red pepper, garlic, green onion, ginger, salted fish and the like, and then ripening the vegetable by lactic acid production and fermenting the ripened vegetable at low temperature. The Lactobacillus paraplantarum CK401 of the present invention is a newly isolated and identified facultative anaerobe.

The present invention also provides an antiviral composition containing any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD, particularly an antiviral composition showing a strong activity against influenza viruses, particularly avian influenza viruses. This composition may be usefully used for drugs, foods, fermented products, food additives, health supplements, feed, feed additives, etc.

Here, the culture fluid contains an active ingredient having antiviral activity, and may be an undiluted culture fluid containing cells, a cell-free culture supernatant, or a diluted culture fluid. The composition may be prepared as a lyophilized form, a freeze-dried form or a hydrated form.

In addition, any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD, have the effect of preventing and treating infection with a wide range of viruses, and preferably exhibit high antiviral activity against influenza viruses, more preferably avian influenza viruses.

The pharmaceutical composition of the present invention refers to a formulation containing any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD, and may be administered together with a pharmaceutically acceptable carrier orally, parenterally, or transdermally or transmucosally through physical contact, or directly into the intestine through a tuber or a catheter.

The pharmaceutical composition may be prepared as a tablet, a capsule, an implant, a suppository, powder, a solution, a gel, a liquid-state suspension, or the like.

In addition, the dose of the pharmaceutical composition may be determined by those skilled in the related technology field, and may vary depending on various factors, including the formulation method, the mode of administration, the subject's age, body weight, sex, disease condition, diet, the duration of administration, the route of administration, excretion rate, and reaction sensitivity.

The fermented food of the present invention may include dairy products that may be refrigerated, frozen or stored for a long period of time, for example, kimchi, milk, milk powder, yogurt, kefir, ice cream, milk shake, cheese, cream, curd, fermented milk, and milk-containing fermented food, as well as soybean milk, fermented grain products, diets for patients, and baby food. The fermented food may also be administered as animal feed to animals.

In order to produce food by culturing the Lactobacillus paraplantarum CK401 of the present invention or add the Lactobacillus paraplantarum CK401 to food, milk or a starch-containing grains, is preferably used as substrates in culturing of the strain.

The composition prepared in the form of food or beverage may include, in addition to the microorganism of the present invention, one or more components selected from among lipids, proteins, carbohydrates, dietary fiber, minerals, and vitamins. Among the above components, particularly the dietary fiber is generally known as a prebiotic substrate that promotes the division of lactic acid bacteria, and thus may play an important role in the formation and maintenance of colonies in the intestines after administration. Accordingly, the dietary fiber is included in the composition of the present invention so that it may be administered simultaneously with or after the composition.

The pharmaceutical composition, health supplement, fermented food or food additive of the present invention may further include a probiotic strain beneficial to the human body, in addition to a pharmaceutically necessary component or a component suitable for food.

The amount of Lactobacillus paraplantarum CK401 contained in the pharmaceutical composition, fermented food or food additive may be about 10⁵ cfu/g to about 10¹¹ cfu/g, preferably 10⁶ cfu/g to about 10¹⁰ cfu/g, more preferably about 10⁷ cfu/g to about 10⁹ cfu/g. For administration, the strain is preferably administered in a viable state, or may be killed before administration or administered in an attenuated state. In addition, where a culture supernatant of the strain is used for production, it may be additionally subjected to a sterilization process through a heat treatment process. The amount or daily intake of the strain necessary to possess a minimum efficacy may vary depending on the physical or health conditions of a subject to be administered with the strain, but is preferably 10⁶ to 10¹² cfu/day, most preferably 10⁷ to 10¹⁰ cfu/day. The degree of antiviral activity of the culture supernatant may be determined using a technique commonly used in the industry and the dose of the culture supernatant may be determined accordingly.

In another aspect, the present invention is directed to a method for preventing influenza viral infection in animals other than humans, the method including administering, to animals other than humans, any one or more selected from the group consisting of Lactobacillus paraplantarum CK401 KCTC 13287BP, a culture fluid of the Lactobacillus paraplantarum CK401 KCTC 13287BP, and a retentate of the culture fluid on a membrane filter having a molecular cutoff of 10 kD.

In the present invention, the method of administrationto animals may be determined by those skilled in the technical field, and may be in the form of a feed additive, a freeze-dried formulation, a diluted cell culture fluid, or the like. In addition, the amount of administration may vary depending on factors such as the mode of feeding, the animal's kind, age, diseased condition, excretion rate and reaction sensitivity.

In the present invention, the animals to be administered may be mammals other than humans, preferably livestock, more preferably poultry.

The Lactobacillus paraplantarum CK401 according to the present invention exhibits a very high antiviral activity when cultured in large amounts by a conventional liquid-state or solid-state culture technology, and thus it is possible to produce a large amount of cells of the microorganism at low costs. Therefore, it is industrially very useful.

Hereinafter, the present invention will be described in more detail with reference to examples. These examples are merely to illustrate the present invention in more detail, and the scope of the present invention is not limited only to these examples.

### Example 1: Isolation of Kimchi Lactobacillus and Production of Antiviral Culture Fluid

Kimchi prepared by a conventional kimchi preparation method was used as a kimchi sample from which Lactobacillus was to be isolated. The kimchi sample was 10-fold diluted with 0.85% saline solution, and 1 ml of the diluted sample solution was inoculated into each MRS agar medium (MRS agar, Difco.; 10 g of bacto peptone, 10 g of beef extract, 5 g of yeast extract, 20 g of glucose, 1 g of Tween 80, 2 g of citric acid, 2 g of dibasic potassium phosphate, 5 g of sodium acetate, 0.1 g of manganese sulfate, 0.05 g of magnesium sulfate, and 15 g of agar per 1 L of distilled water), and then streaked with a glass rod. Thereafter, the plate was cultured in a constant-temperature incubator at 30° C. for 48 hours. Each of the produced colonies was streaked on MRS agar medium and cultured at 30° C. for 48 hours. Eight colonies considered dominant based on the type, color, and the like of the grown colonies were selected and secondarily cultured on MRS medium. Thereafter, whether or not the bacteria to be isolated would be single strains was checked.

Eight kinds of kimchi-derived Lactobacillus strains selected as single strains from the secondary culture were each inoculated into 50 ml of MRS broth (Difco) and cultured stationarily at 30° C. for 24 hours. The resulting cultured fluids were centrifuged at 6000 rpm for 5 minutes to remove the cells, and were then neutralized to a pH of 6.5 and filtered through 0.22 μm, thereby producing lactobacillus culture fluids.

### Example 2: Evaluation of the Antiviral Activities of Eight Kinds of Kimchi-Derived Lactobacillus Strains Selected as Single Strains

The antiviral activities of the eight kinds of lactobacillus strains selected in Example 1 were tested in the following method. As a virus, A/CK/ANYANG/MS96 (H9N2) was used, which is a low-pathogenic avian influenza H9N2 serotype virus isolated in Korea and characterized. The virus AIV MS96 strain was cultured in a 10 to 11-day-old SPF embryonated egg and proliferated to reach a titer of at least 10^{7.5} EID₅₀/ml. The proliferated virus was stored in a freezer maintained at −70° C. during use.

The allantoic fluid containing the viral solution was mixed with each lactobacillus strain sample in the same amounts (1:1), followed by sensitization at 25° C. for 45 minutes. 0.1 ml of the resulting reaction solution was inoculated into three 10 to 11-day-old SPF embryonated eggs by a 1 cc syringe. A uninoculated control group and a positive control group were also cultured, and 1 mg/ml of Oseltamivir phosphate (Tamiflu, Roche) was used as a control sample for the eight kinds of kimchi-derived lactobacillus strains. After the inoculation, incubation was performed at 37° C. for 5 days, and during the incubation, egg inspection was performed daily and the embryonated eggs that died within 24 hours after the inoculation was regarded as an accidental death and excluded from the test results. The inoculated eggs that died within 5 days from 24 hours after the inoculation were all stored at 4° C. Allantoic fluids were collected respectively from all the embryonated eggs that survived even after 5 days after the inoculation and the dead inoculated eggs stored at 4° C., and a hemagglutination test and a titer test were performed on the collected allantoic fluids to determine whether or not the virus would exist in the allantoic fluids.

As shown in Table 1 below, the results of the test showed that in the case of CK-401 among the eight kinds of lactobacillus strains, the virus was not detected in all the three eggs sensitized and inoculated with the virus. That is, no hemagglutination ability was observed in the allantoic fluids of the inoculated eggs. It could be seen that the eggs of the negative control group for incubation all survived, indicating that there was no problem in the incubation during the experiment, and the eggs of the positive control group inoculated with the virus alone all showed hemagglutination ability, indicating that the virus was correctly inoculated. Tamiflu used as the control drug also showed antiviral activity. As a result, it was confirmed that only CK-401 had an antiviral effect comparable with that of commercially available Tamiflu.

TABLE 1

| | Negative | Positive | | Samples (CK) | | | | | | | |
|---|---|---|---|---|---|---|---|---|---|---|---|
| | control | control | Tamiflu | 101 | 201 | 301 | 401 | 501 | 601 | 701 | 801 |
| Sample | — | — | Tamiflu | yes | yes | yes | yes | yes | yes | yes | yes |
| Virus | — | yes | yes | yes | yes | yes | yes | yes | yes | yes | yes |
| HA | 0/3 | 3/3 | 0/3 | 3/3 | 3/3 | 3/3 | 0/3 | 3/3 | 3/3 | 3/3 | 3/3 |
| positive | | | | | | | | | | | |
| HA titer | 0 | 7 to | 0 | 7 to | 7 | 8 to | 0 | 6 to | 7 to | 7 | 7 |
| | | 9 | | 9 | | 11 | | 7 | 11 | | |

### Example 3: Evaluation of Antiviral Activity of Lactobacillus paraplantarum CK401 Against Avian Influenza

The antiviral activity of the CK-401 lactobacillus strain selected in Example 2 was tested in the following method. As a virus, A/CK/ANYANG/MS96 (H9N2) was used, which is a low-pathogenic avian influenza H9N2 serotype virus isolated in Korea and characterized. The virus AIV MS96 strain was cultured in a 10 to 11-day-old SPF embryonated egg and proliferated to reach a titer of at least 10^{7.5} EID₅₀/ml. The proliferated virus was stored in a freezer maintained at −70° C. during use. The lactobacillus culture fluid was serially diluted 10-fold with distilled water and maintained at 4° C. before use.

1.0 ml of an allantoic fluid containing a10-fold diluted viral solution was mixed with 24 ml of distilled water, thereby preparing a virus for reaction with the lactobacillus culture fluids. 2.5 ml of the lactobacillus culture fluid serially diluted with distilled water was introduced into a test tube, and 2.5 ml of the prepared viral solution is introduced into the test tube and mixed therewith (a total of 5 ml), followed by sensitization at 25° C. for 45 minutes. After completion of the reaction, in order to determine whether or not the virus would proliferate, the resulting reaction solution was diluted to 10⁻¹, 10⁻², 10⁻³, 10⁻⁴, 10⁻⁵ and 10⁻⁶ with PBS, and 0.2 ml of the neutralized reaction solution was inoculated into the allantoic cavities of five 10-day-old embryonated eggs per dilution rate. After the inoculation, inoculation was performed incubation was performed at 37° C. for 5 days, and during the incubation, egg inspection was performed daily and the embryonated eggs that died within 24 hours after the inoculation was regarded as an accidental death and excluded from the test results. The inoculated eggs that died within 5 days from 24 hours after the inoculation were all stored at 4° C. Allantoic fluids were collected respectively from all the embryonated eggs that survived even after 5 days after the inoculation and the dead inoculated eggs stored at 4° C., and a hemagglutination test was performed on the collected allantoic fluids to determine whether or not the virus would exist in the allantoic fluids.

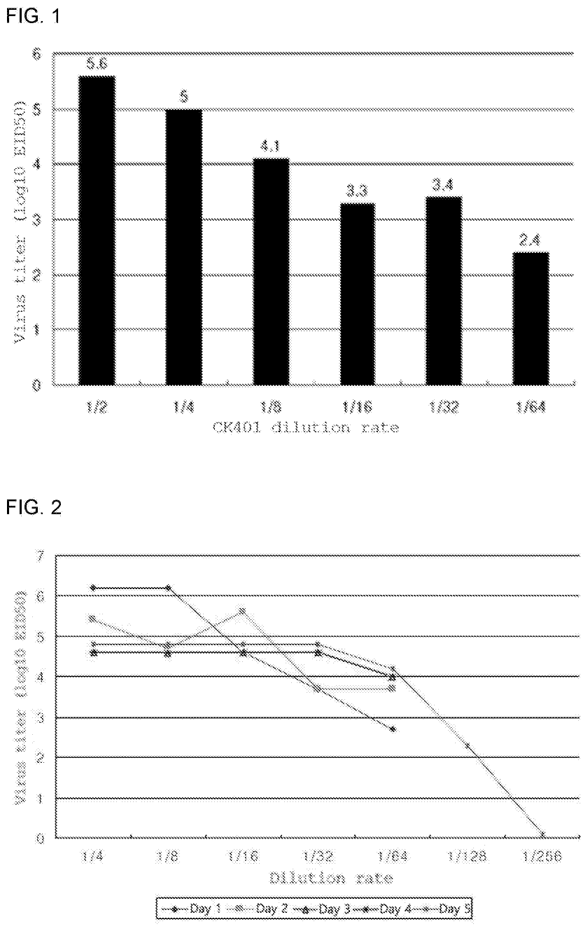

As a result, it could be confirmed that the control group showed a virus titer of 10^{5.6}, whereas the eggs treated with the lactobacillus culture fluid of the CK-401 strain showed virus titers of 0, 10^{0.6} and 10^{1.5} at dilution rates of 1/2, 1/4 and 1/8, respectively, and also showed a virus titer of 10^{3.2} even at a dilution rate of 1/64, suggesting that the lactobacillus culture fluid has a very high antiviral activity (Table 2 and FIG. 1).

TABLE 2

| | | | | | | | | Virus | Viral |
|---|---|---|---|---|---|---|---|---|---|
| | | Degree of AIV killed after treatment | | | | | | content | decrement |
| | | (Number of AIV-positive eggs/ | | | | | | (EID₅₀/ | (EID₅₀) |
| Lactobacillus | | number of inoculated eggs) | | | | | | 0.2 | compared |
| culture fluid | pH | 10⁻¹ | 10⁻² | 10⁻³ | 10⁻⁴ | 10⁻⁵ | 10⁻⁶ | ml) | to control |
| CK401 1/2 | 3.12 | 0/5 | 0/5 | 0/5 | 0/5 | 0/5 | 0/5 | 0 | 10^{5.6} |
| 1/4 | 3.14 | 1/5 | 0/5 | 0/5 | 0/5 | 0/5 | 0/5 | 10^{0.6} | 10^{5.0} |
| 1/8 | 3.16 | 5/5 | 0/5 | 0/5 | 0/5 | 0/5 | 0/5 | 10^{1.5} | 10^{4.1} |
| 1/16 | 3.18 | 5/5 | 3/5 | 1/5 | 0/5 | 0/5 | 0/5 | 10^{2.3} | 10^{3.3} |
| 1/32 | 3.22 | 5/5 | 3/5 | 0/5 | 0/5 | 0/5 | 0/5 | 10^{2.2} | 10^{3.4} |
| 1/64 | 3.30 | 5/5 | 5/5 | 3/5 | 0/5 | 0/5 | 0/5 | 10^{3.2} | 10^{2.4} |
| Control virus | — | 5/5 | 5/5 | 5/5 | 5/5 | 5/5 | 1/5 | 10^{5.6} | — |

### Example 4: Identification of Lactobacillus paraplantarum CK401 Strain

The strain CK401 was isolated from kimchi produced by a conventional kimchi production method, and the strain was identified to be Lactobacillus paraplantarum based on 16S rRNA nucleotide sequencing. The nucleotide sequence was deposited with the GenBank on Jun. 13, 2017 under accession number KCTC13287BP. This strain is currently stored in the Korean Collection for Type Cultures (KCTC), Korea Research Institute of Bioscience and Biotechnology, in the name of Lactobacillus paraplantarum CK401 (the Korean Collection for Type Cultures, Korea Research Institute of Bioscience and Biotechnology, KCTC 13287 BP).

### Example 5: Evaluation of the Avian Influenza Antiviral Activity of Lactobacillus paraplantarum CK401 Strain Following Mass Culture

After the antiviral activity of the Lactobacillus paraplantarum CK401 strain was confirmed, for mass culture, the strain was seed cultured in a flask. Thereafter, the seed strain was transferred into a mass culture chamber, and when there were a decrease in pH, an increase in OD and a decrease in DO at about 12 hours after the start of seed culture, the strain was transferred into a main culture tank.

In main culture, the previously seed-cultured Lactobacillus paraplantarum CK401 strain was inoculated into a liquid-state medium (75 kg of yeast extract, 30 kg of ammonium citrate (NH₄)₃C₆H₅O₇), 75 kg of sodium acetate, 750 g of manganese sulfate (MnSO₄), 30 kg of dipotassium phosphate, and 3 kg of a defoaming agent) sterilized with high-pressure steam, and was cultured under 30° C. and 30 rpm.

The strain was sampled at varying time points during the culture, and the lactobacillus cell number, pH, OD and glucose level of the sample were analyzed. In addition, on each day of culture, the avian influenza antiviral activity of the strain was analyzed in the same manner as Example 3.

As a result, it was confirmed that the group treated with the culture fluid mass-cultured for 3 to 5 days rather than the culture fluid mass-cultured for 1 to 2 days showed a viral titer of 0 at a dilution rate of up to 1/32, compared to the control virus. Thus, it was found that the effective concentration of the 3 to 5-day culture fluid was higher than that of the 1 to 2-day culture fluid (Tables 3, 4 and 5 and FIG. 2).

Accordingly, it is considered that the CK-401 lactobacillus strain can be mass-cultured, has excellent antiviral activity, and thus is industrially very useful.

TABLE 3

| | | | | | | | | Virus | Viral |
|---|---|---|---|---|---|---|---|---|---|
| | | Degree of AIV killed after treatment | | | | | | content | decrement |
| Culture | | (Number of AIV-positive eggs/ | | | | | | (EID₅₀/ | (EID₅₀) |
| period | Treatment | inoculated eggs at each dilution rate) | | | | | | 0.2 | compared |
| (days) | concentration | 10⁻¹ | 10⁻² | 10⁻³ | 10⁻⁴ | 10⁻⁵ | 10⁻⁶ | ml) | to control |
| 1 | 1/4 | 0/5 | 0/5 | 0/5 | 0/5 | 0/5 | 0/5 | 0 | 10^{6.2} |
| | 1/8 | 0/5 | 0/5 | 0/5 | 0/5 | 0/5 | 0/5 | 0 | 10^{6.2} |
| | 1/16 | 5/5 | 0/5 | 0/5 | 0/5 | 0/5 | 0/5 | 10^{1.6} | 10^{4.6} |
| | 1/32 | 5/5 | 5/5 | 0/5 | 0/5 | 0/5 | 0/5 | 10^{2.5} | 10^{3.7} |
| | 1/64 | 5/5 | 5/5 | 5/5 | 5/5 | 0/5 | 0/5 | 10^{3.5} | 10^{2.7} |
| 2 | 1/4 | 2/5 | 0/5 | 0/5 | 0/5 | 0/5 | 0/5 | 10^{0.8} | 10^{5.4} |
| | 1/8 | 5/5 | 0/5 | 0/5 | 0/5 | 0/5 | 0/5 | 10^{1.3} | 10^{4.7} |
| | 1/16 | 1/5 | 0/5 | 0/5 | 0/5 | 0/5 | 0/5 | 10^{0.6} | 10^{5.6} |
| | 1/32 | 5/5 | 5/5 | 0/5 | 0/5 | 0/5 | 0/5 | 10^{2.5} | 10^{3.7} |
| | 1/64 | 5/5 | 5/5 | 0/5 | 0/5 | 0/5 | 0/5 | 10^{2.5} | 10^{3.7} |
| Control | — | 5/5 | 5/5 | 5/5 | 5/5 | 5/5 | 3/5 | 10^{6.2} | — |
| virus | | | | | | | | | |

TABLE 4

| | | | | | | | | Virus | Viral |
|---|---|---|---|---|---|---|---|---|---|
| | | Degree of AIV killed after treatment | | | | | | content | decrement |
| Culture | | (Number of AIV-positive eggs/ | | | | | | (EID₅₀/ | (EID₅₀) |
| period | Treatment | inoculated eggs at each dilution rate) | | | | | | 0.2 | compared |
| (days) | concentration | 10⁻¹ | 10⁻² | 10⁻³ | 10⁻⁴ | 10⁻⁵ | 10⁻⁶ | ml) | to control |
| 3 | 1/4 | 0/5 | 0/5 | 0/5 | 0/5 | 0/5 | 0/5 | 0 | 10^{4.6} |
| | 1/8 | 0/5 | 0/5 | 0/5 | 0/5 | 0/5 | 0/5 | 0 | 10^{4.6} |
| | 1/16 | 0/5 | 0/5 | 0/5 | 0/5 | 0/5 | 0/5 | 0 | 10^{4.6} |
| | 1/32 | 0/5 | 0/5 | 0/5 | 0/5 | 0/5 | 0/5 | 0 | 10^{4.6} |
| | 1/64 | 1/5 | 0/5 | 0/5 | 0/5 | 0/5 | 0/5 | 10^{0.6} | 10^{4.0} |
| 4 | 1/4 | 0/5 | 0/5 | 0/5 | 0/5 | 0/5 | 0/5 | 0 | 10^{4.6} |
| | 1/8 | 0/5 | 0/5 | 0/5 | 0/5 | 0/5 | 0/5 | 0 | 10^{4.6} |
| | 1/16 | 0/5 | 0/5 | 0/5 | 0/5 | 0/5 | 0/5 | 0 | 10⁴⁶ |
| | 1/32 | 0/5 | 0/5 | 0/5 | 0/5 | 0/5 | 0/5 | 0 | 10^{4.6} |
| | 1/64 | 1/5 | 0/5 | 0/5 | 0/5 | 0/5 | 0/5 | 10^{0.6} | 10^{4.0} |
| Control | — | 5/5 | 5/5 | 5/5 | 5/5 | 1/5 | 0/5 | 10^{4.6} | — |
| virus | | | | | | | | | |

TABLE 5

| | | | | | | | | Virus | Viral |
|---|---|---|---|---|---|---|---|---|---|
| | | Degree of AIV killed after treatment | | | | | | content | decrement |
| culture | | (Number of AIV-positive eggs/ | | | | | | (EID₅₀/ | (EID₅₀) |
| period | Treatment | inoculated eggs at each dilution rate) | | | | | | 0.2 | compared |
| (days) | concentration | 10⁻¹ | 10⁻² | 10⁻³ | 10⁻⁴ | 10⁻⁵ | 10⁻⁶ | ml) | to control |
| 5 | 1/4 | 0/5 | 0/5 | 0/5 | 0/5 | 0/5 | 0/5 | 0 | 10^{4.8} |
| | 1/8 | 0/5 | 0/5 | 0/5 | 0/5 | 0/5 | 0/5 | 0 | 10^{4.8} |
| | 1/16 | 0/5 | 0/5 | 0/5 | 0/5 | 0/5 | 0/5 | 0 | 10^{4.8} |
| | 1/32 | 0/5 | 0/5 | 0/5 | 0/5 | 0/5 | 0/5 | 0 | 10^{4.8} |
| | 1/64 | 1/5 | 0/5 | 0/5 | 0/5 | 0/5 | 0/5 | 10^{0.6} | 10^{4.2} |
| | 1/128 | 5/5 | 0/5 | 0/5 | 0/5 | 0/5 | 0/5 | 10^{2.5} | 10^{2.3} |
| | 1/256 | 5/5 | 5/5 | 5/5 | 4/5 | 2/5 | 0/5 | 10^{4.7} | 10^{0.1} |
| Control | — | 5/5 | 5/5 | 5/5 | 5/5 | 5/5 | 0/5 | 10^{4.8} | — |
| virus | | | | | | | | | |

### Example 6: Field Test for Lactobacillus paraplantarum CK401 Strain

After the safety and efficacy of the strain were examined in the laboratory test, an field test on the strain was performed to evaluate the stability of the strain in an actual outdoor field and the field applicability of the strain.

(1) Test Method

1) Test Animals and Inoculation

Three commercial layer flocks located in geographically separated districts were selected and inoculated with a culture fluid of the Lactobacillus paraplantarum CK401 strain according to the present invention. Farms were randomly selected within the districts, but farms satisfying the following requirements were used as selection objects: records should be faithful due to the nature of the experiment; henhouses located in each farm should have the same or similar sizes; layer flocks should be as uniform as possible; and the size of each henhouse should be at least 2,000 hens.

In the farms, the test substance was administered through an automatic water feeder so that it could be administered as continuously as possible, or the test substance was added to a water bucket.

2) Serological Test

For the flocks of the selected farms, as an antibody test for low-pathogenic avian influenza according to the purpose of this study, a hemagglutination inhibition (HI) test was performed periodically. In addition, according to the farm's own needs, antibody tests for Newcastle disease and infectious bronchitis were basically performed. For avian influenza and Newcastle disease, an HI test was performed, and for infectious bronchitis, a test was performed using an ELISA kit imported from IDEXX Inc. (USA).

3) Egg Production Rate and Abnormal Egg Production Rate

In order to analyze safety as the purpose of this experiment, egg production rate which is the most sensitive factor in laying hen farms was examined daily after administration of the test sample, and the examination was conducted for about 2 months depending on the farm's situation. The egg production rate was examined by distinguishing between a group administered with the test sample and a non-administered group, and when there were abnormalities in egg production rate was abnormal, sampling was performed for diagnosis. When there were abnormalities in eggshell or egg quality, serum tests for infectious diseases (i.e., Newcastle disease and infectious bronchitis), which cause abnormalities in eggshell or egg quality, were performed to evaluate the influence of the test sample.

4) Viral Isolation

Although periodic serum tests were performed to identify the farm infection with avian influenza, which is the most important factor in this experiment, viral isolation or genetic search was performed directly to confirm the infection more reliably.

5) Outdoor Farms Inoculated with Culture Fluid of CK401 Strain According to the Present Invention

TABLE 6

| | | | | | Number | Number | |
|---|---|---|---|---|---|---|---|
| | | Kind | | Chick | of | of | |
| | | of | | placement | chicken | chicken | Type of |
| Farms | Districts | chicken | Variety | date | raised | tested | henhouse |
| PC | Cheonan- | Laying | Lohman | Sep. 10, | 8,000 | 8,000 | Windowless |
| | si, Chungcheongnam- | hens | | 2005 | | | |
| | do, Korea | | | Apr. 2, | 10,000 | 10,000 | Windowless |
| | | | | 2005 | | | |
| SH | Cheonan- | Laying | Hy-Line | May 6, | 20,000 | 20,000 | With |
| | si, Chungcheongnam- | hens | | 2005 | | | window |
| | do, Korea | | | Dec. 10 | 5,000 | 5,000 | With |
| | | | | 2004 | | | window |
| CW | Cheonan- | Laying | Hy-Line | Aug. 19, | 11,880 | 11,880 | Windowless |
| | si, Chungcheongnam- | hens | | 2005 | | | |
| | do, Korea | | | Aug. 19, | 11,722 | 11,722 | Windowless |
| | | | | 2005 | | | |

As shown in Table 6 above, three farms located in Cheonan-si, Chungcheongnam-do, Korea were selected. The actual distance between the farms was more than 10 km. Two farms had windowless henhouses, and one farm had henhouses with windows. One of the farms was a farm with excellent facilities and management, one was a farm with moderate facilities and management, and one was a farm with somewhat poor facilities and management. Regular visits to the farms were made, and in fact, the SH farm among the farms had a history of infection with avian influenza, and for this reason, was recently damaged.

(2) Results of Inoculation with CK401 Strain Culture Fluid in PC Outdoor Farm

In the inoculated group, eggshell abnormalities, which were presumed to be caused by stress at the initial stage of the test after inoculation, occurred briefly, but disappeared immediately after the use of respiratory-related antibiotics. This phenomenon is a phenomenon that generally occurs when a new drug is fed, and it was found that this phenomenon had no particular problem. In subsequent continuous inoculation with the test sample, symptoms, such as diarrhea that appears upon drinking water inoculation, did not appear, and thus it was considered that there was no respiratory or digestive disorder and that the test sample was safe.

1) Egg Production Rate and Abnormal Egg Production Rate

The egg production rate of the PC farm was examined by distinguishing between an inoculated group and a control group. It was generally observed that the egg production rate of the inoculated group was lower than that of the control group, but this is because the age of the hens did differ between the two groups. That is, it is considered that this phenomenon appeared due to a difference of five months in the time of chick placement, because the inoculated group was a group of chicks placed on April 2015 and the control group was a group of chicks placed on September 2015. In fact, in the case of the inoculated group, the egg production rate at the time of inoculation decreased slowly, even though the egg production rate would necessarily continue to decrease. Thus, the farmer considered that the egg production rate increased rather than decreased (see FIG. 1).

2) Change in Serum Titer

TABLE 7

| | | Laying | | | |
|---|---|---|---|---|---|
| Name of | Day of | hen | Days after KC inoculation | | |
| disease | inoculation | groups | 0 | 18 | 41 |
| AI | | Inoculated | 0 | 0 | 0 |
| | | group | | | |
| | | Control | 0 | 0 | 0 |
| | | group | | | |
| ND | | Inoculated | 7.9 ± 0.8 | 10.8 ± 0.4 | 9.4 ± 1.3 |
| | | group | | | |
| | | Control | 8.2 ± 1.2 | 9.2 ± 0.4 | 9.6 ± 0.8 |
| | | group | | | |
| IB | | Inoculated | 9938 ± 2699 | 3158 ± 2523 | 4241 ± 3748 |
| | | group | | | |
| | | Control | 9560 ± 2269 | 5959 ± 2179 | 8222 ± 2669 |
| | | group | | | |

As can be seen in Table 7 above, the results of the antibody test for avian influenza (AI) showed that no antibody was detected during the test period, suggesting that there was no infection in the actual outdoor field.

In addition, there was no distinct difference in the Newcastle disease (ND) antibody between the inoculated group and the control group, suggesting that inoculation of the test sample fundamentally had no influence. In an antibody test for infectious bronchitis (IB), the level of ELISA titer should be a certain level (ELISA titer level: 10,000) or more so that interpretation about outdoor infection or vaccination is possible. In general, since outdoor field infection should correspond to an ELISA titer of 10,000 or more, it was considered that there was no outdoor field infection of the inoculated group as the IB titers of the inoculated groups were all at low levels. In addition, it was confirmed that the test substance caused no side effect.

3) Viral Isolation

After inoculation of the test substance, a test for low-pathogenic avian influenza infection can be performed by the HI test, but the HI test has disadvantage that it cannot detect various serotypes and takes time to form an antibody after infection. Hence, viral isolation tests for the farms were performed.

TABLE 8

| Days | | Viral samples | | |
|---|---|---|---|---|
| after | | Bronchial | | |
| inoculation | Groups | tube | Feces | Others |
| 18 | Inoculated group | 0/5 | 0/5 | — |
| | Control group | 0/5 | 0/5 | — |
| 41 | Inoculated group | 0/5 | 0/5 | — |
| | Control group | 0/5 | 0/5 | — |

As can be seen in Table 8 above, on 18 and 41 days after inoculation, feces samples (10 feces swab samples per tube), bronchial samples (5 swab samples per tube) and cloaca samples (5 swab samples per tube) were collected and sent to the laboratory in which a viral isolation test and a genetic test (PCR) were performed.

It was confirmed that neither viral isolation nor avian influenza virus gene was detected in all the bronchial and feces samples during the test period, and thus there was no outdoor field infection.

(3) Results of Inoculation with CK401 Strain Culture Fluid in SH Outdoor Farm

After administration of the test substance, no clinical symptoms related to respiratory or digestive organs were observed. The SH farm has experienced several avian influenza outbreaks in the past, and hence intensive observation of clinical symptoms associated with, particularly, avian influenza was performed, but it was confirmed that no particular problem arose. In addition, because the test sample is fed through drinking water, digestive disorders may occur, and thus observation about these disorders was performed. No specific symptom appeared during the test period, and thus it was finally determined that administration of the test sample cause no side effect and is safe.

1) Egg Production Rate and Abnormal Egg Production Rate

The egg production rate of the SH farm was examined by distinguishing between an inoculated group and a control group.

It was generally observed that there was little difference in egg production rate between the inoculated group and the control group (see FIG. 2).

2) Change in Serum Titer

TABLE 9

| | | Laying | | | |
|---|---|---|---|---|---|
| Name of | Day of | hen | Days after KC inoculation | | |
| disease | inoculation | groups | 0 | 18 | 41 |
| AI | | Inoculated | 0 | 0 | 0 |
| | | group | | | |
| | | Control | 0 | 0 | 0 |
| | | group | | | |
| ND | | Inoculated | 8.4 ± 07* | 10.4 ± 0.4 | 8.9 ± 1.2 |
| | | group | | | |
| | | Control | 9.4 ± 0.5 | 11.2 ± 0.4 | 10.5 ± 1.1 |
| | | group | | | |
| IB | | Inoculated | 5958 ± 3432 | 5174 ± 3200 | 9141 ± 2395 |
| | | group | | | |
| | | Control | 8869 ± 2411 | 5185 ± 2596 | 8386 ± 1609 |
| | | group | | | |

*log2

As can be seen in Table 9 above, the results of antibody test for avian influenza (AI) showed that no antibody was detected during the test period, suggesting that there was no infection in the actual outdoor field.

In addition, there was no distinct difference in the Newcastle disease (ND) antibody between the inoculated group and the control group, suggesting that inoculation of the test sample fundamentally had no influence.

In an antibody test for infectious bronchitis (IB), the level of ELISA titer should be a certain level (ELISA titer level: 10,000) or more so that interpretation about outdoor infection or vaccination is possible. In general, since outdoor infection should correspond to an ELISA titer of 10,000 or more, it was considered that there was no outdoor infection of the inoculated group as the IB titers of the inoculated group were all at low levels. In addition, it was confirmed that the test substance caused no side effect.

3) Viral Isolation

TABLE 10

| | | Viral samples | | |
|---|---|---|---|---|
| Days after | | Bronchial | | |
| inoculation | Groups | tube | Feces | Others |
| 18 | Inoculated group | 0/5 | 0/5 | — |
| | Control group | 0/5 | 0/5 | — |
| 41 | Inoculated group | 0/5 | 0/5 | — |
| | Control group | 0/5 | 0/5 | — |

As can be seen in Table 10 above, on 18 and 41 days after inoculation, feces samples (10 feces swab samples per tube), bronchial samples (5 swab samples per tube) and cloaca samples (5 swab samples per tube) were collected and sent to the laboratory in which a viral isolation test and a genetic test (PCR) were performed.

It was confirmed that neither viral isolation nor avian influenza virus gene was detected in all the bronchial and feces samples during the test period, and thus there was no outdoor infection.

(4) Results of Inoculation with CK401 Strain Culture Fluid in CW Outdoor Farm

After administration of the test substance, clinical symptoms related to the respiratory or digestive organs were not observed, it was shown that the number of laid eggs having much feces on the eggshell increased. In general, the reason why much feces is on the eggshell is believed to be because the content of water or mucous substance in the feces is more than that on normal eggs. This phenomenon disappeared afterwards, and thus it was considered that this phenomenon was caused by temporary stress appearing at the beginning of the inoculation. However, in the case of this farm, the egg color was not good in many cases, and it was shown that after administration of the test sample, the egg color of the inoculated group became rather better than that of the control group. Therefore, it was concluded that administration of the test substance in the CW farm had no safety problem.

1) Egg Production Rate and Abnormal Egg Production Rate

The egg production rate in the CW farm was examined by distinguishing between an inoculated group and a control group. In the case of the PC farm and the SH farm, the daily egg production rates were recorded and compared, but in the case of the CW farm, at weeks 4 and 5, weekly records were prepared without preparing daily records, and all the records were converted to weekly records and analyzed. Looking at the egg production rate, it could be seen that at 3 weeks after administration, the egg production rate of the control group was declined compared to that of the inoculated group, and after 1 week, it was recovered again.

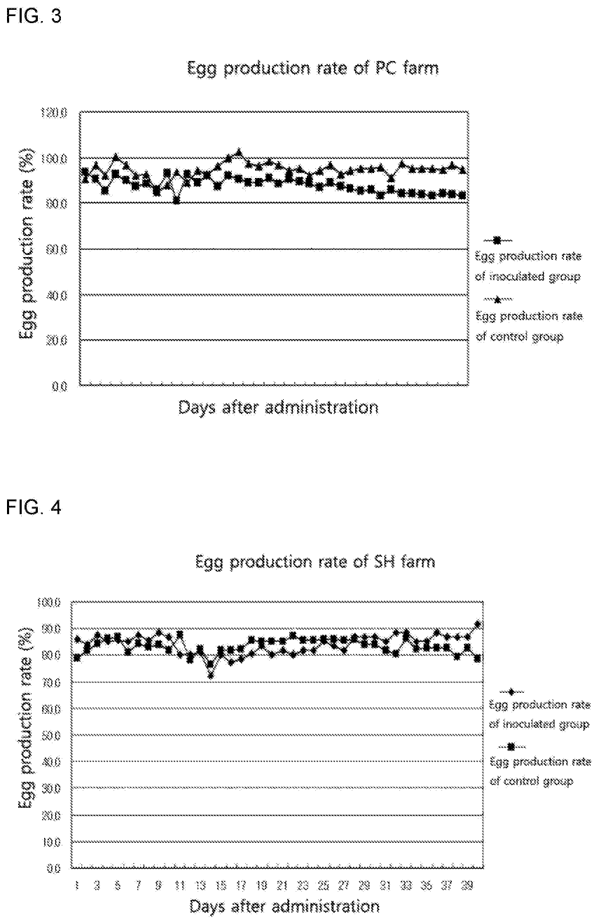

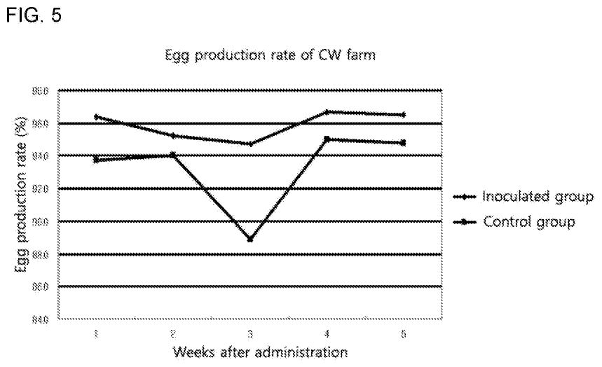

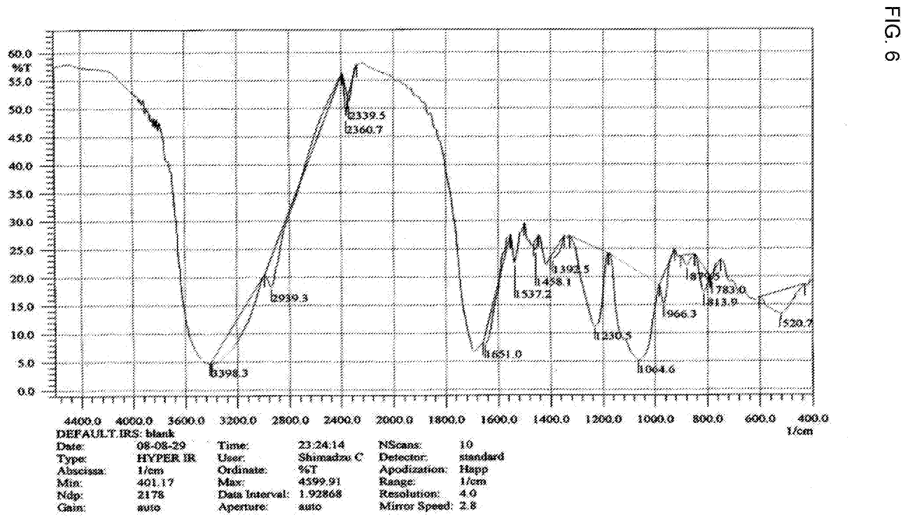

This phenomenon was generally considered caused by outdoor infection with infectious disease, and when an actual serum test was performed, it could be observed that the titer of infectious bronchitis increased in the control group during the test period. However, within the same period, a change in the egg production rate or a rapid change in the infectious bronchitis antibody titer was not observed in the infected group, suggesting that the inoculated group was not infected with infectious disease (see FIG. 3).

2) Change in Serum Titer

TABLE 11

| Name | Day of | Laying | | | |
|---|---|---|---|---|---|
| of | inoc- | hen | Days after KC inoculation | | |
| disease | ulation | groups | 0 | 18 | 41 |
| AI | | Inoculated | 0 | 0 | 0 |
| | | group | | | |
| | | Control | 0 | 0 | 0 |
| | | group | 5.8 ± 0.6 | 8.6 ± 0.7 | 6.4 ± 0.5 |
| ND | | Inoculated | | | |
| | | group | | | |
| | | Control | 6.3 ± 1.2 | 7.3 ± 1.3 | 7.2 ± 0.4 |
| | | group | | | |
| IB | | Inoculated | 9146 ± 2508 | 7652 ± 4074 | 7420 ± 3047 |
| | | group | | | |
| | | Control | 9287 ± 2639 | 3198 ± 2506 | 10438 ± 2896 |
| | | group | | | |

As can be seen in Table 11 above, the results of antibody test for avian influenza (AI) showed that no antibody was detected during the test period, suggesting that there was no infection in the actual outdoor field.

In addition, there was no distinct difference in the Newcastle disease (ND) antibody between the inoculated group and the control group, suggesting that inoculation of the test sample fundamentally had no influence.

The titer of the antibody for infectious bronchitis (IB) increased in the control group at 41 days after inoculation, and the titer at this time was sufficient to be recognized as outdoor infection. The titer of the antibody in the inoculated group was at the antibody titer level of normal laying hens and was difficult to consider as infection. When making a simple judgment based on the titer, there was infectious bronchitis in the control group, and there was no change in the antibody titer in the inoculated group, even though whether or not the inoculated group would be infected could not be determined. Considering the above-described egg production results, it was determined that only the control group was affected by infectious bronchitis.
3) Viral Isolation

TABLE 12

| | | Viral samples | | |
|---|---|---|---|---|
| Days after | | Bronchial | | |
| inoculation | Groups | tube | Feces | Others |
| 18 | Inoculated group | 0/5 | 0/5 | — |
| | Control group | 0/5 | 0/5 | — |
| 41 | Inoculated group | 0/5 | 0/5 | — |
| | Control group | 0/5 | 0/5 | — |

As can be seen in Table 12 above, on 18 and 41 days after inoculation, feces samples (10 feces swab samples per tube), bronchial samples (5 swab samples per tube) and cloaca samples (5 swab samples per tube) were collected and sent to the laboratory in which a viral isolation test and a genetic test (PCR) were performed.

It was confirmed that neither viral isolation nor avian influenza virus gene was detected in all the bronchial and feces samples during the test period, and thus there was no outdoor infection.

### Example 7: Analysis of Molecular Weight-Dependent Antiviral Activity of Culture Fluid of Lactobacillus paraplantarum CK401

The lactobacillus culture fluid of Example 3 was filtered through a membrane filter having a molecular weight cutoff of 10 kD, and then the antiviral activity of each of the permeate and retentate of the filter was tested in the same method as Example 3. The results are shown in Table 13 below.

TABLE 13

| | | Degree of AIV killed after treatment | | | | | |
|---|---|---|---|---|---|---|---|
| | | (Number of AIV positive eggs/number of inoculated eggs) | | | | | |
| Test sample | | 1/2 D | 1/4 D | 1/8 D | 1/16 D | 1/32 D | Remarks |
| 10 | Permeate of | 5/5 | 5/5 | 5/5 | 5/5 | 5/5 | No |
| KFT | 10 kD | | | | | | virucidal |
| | membrane | | | | | | |
| 10 | Retentate of | | | | | | |
| KRE | 10 kD | 0/5 | 0/5 | 0/5 | 0/5 | 3/5 | Vimcidal |
| | membrane | | | | | | |

Referring to Table 13 above, it could be confirmed that the permeate through the membrane filter having a molecular weight cutoff of 10 kD showed no antiviral activity, whereas the retentate showed antiviral activity in all the cases in which it was diluted 1/2 to 1/32.

### Example 8: Identification of 10-kD Membrane Filter Retentate Having Antiviral Activity

The antiviral active substance of Example 7 was subjected to an antiviral test after 2 hours of heating at 80° C. and an antiviral test after adding 0.1% SDS. As a result, it was confirmed that the antiviral active substance

Showed the antiviral activity in all the antiviral tests, suggesting that the retentate was not protein. In addition, FT-IR analysis was performed to confirm the main skeleton of the retentate, and the results are shown in FIG. 6. As a result of reviewing FIG. 6, it was determined that the main component of the 10-kD membrane filter retentate is highly likely to be a polysaccharide.

The reason for the above determination is that the main absorption bands appearing at 3402 cm⁻¹ (—OH), 2937 cm⁻¹ (—CH2), 1647 cm⁻¹ (associated water) and 1053 cm⁻¹ (—C—O—C—) and the patterns of the bands were similar to those of the exopolysaccharide produced by lactobacillus, and that the absorption band observed at 1647 cm⁻¹ corresponds to associated water contained in the carbohydrate itself.

1-8. (canceled)

9. Lactobacillus paraplantarum CK401 KCTC 13287BP a producing culture fluid with an antiviral activity against avian influenza virus.

10. An antiviral composition against avian influenza virus containing a retentate of a membrane filter having a molecular weight cutoff of 10 kD in culture fluid of Lactobacillus paraplantarum CK401 KCTC 13287BP.

11. A method for preventing and treating avian influenza viral infection in animals other than humans, the method comprising administering, to animals other than humans, a retentate of a membrane filter having a molecular weight cutoff of 10 kD in culture fluid of Lactobacillus paraplantarum CK401 KCTC 13287BP.
